(19) United States
(12) Patent Application Publication
(10) Pub. No.: US 2015/0305622 A1
(43) Pub. Date: Oct. 29, 2015

(54) Optical Low Coherence Microphone

(71) Applicant: Joshua Noel Hogan, Los Altos, CA (US)

(72) Inventors: Joshua N. Hogan, Los Altos, CA (US); Sergey Alexandrov, Galway (IE)

(21) Appl. No.: 14/604,745

(22) Filed: Jan. 25, 2015

Related U.S. Application Data
(60) Provisional application No. 61/931,854, filed on Jan. 27, 2014.

(51) Int. Cl. A61B 5/00; H04R 3/00; G01H 9/00; H04R 1/08

(52) U.S. Cl. CPC A61B 5/0066; H04R 1/08; H04R 3/00; G01H 9/00; A61B 5/7278; A61B 2576/00

(57) ABSTRACT

A micro phonic device capable of measuring very low frequency (sub 100 Hz) pressure waves includes an OCT measuring system that measures the position of one or more surfaces within a target. The device further processes the position measurements to generate a spectrum. In one application the generated spectrum is further processed to associate some signals with bio-sign parameters. Values of the bio-sign parameters are output.

### CROSS REFERENCES TO RELATED APPLICATIONS

This utility application, docket number CI131201US claims priority from provisional application numbered 61/931,854 (docket number CI131201PR); and relates to U.S. Pat. No. 7,526,329 filed on Dec. 29, 2004 titled “Multiple Reference Non-invasive Analysis System” and U.S. Pat. No. 7,751,862, filed on Jan. 31, 2005 titled “Frequency Resolved Imaging System”, and U.S. Pat. No. 8,605,290, filed on May 23, 2010 titled “Precision Measuring System” the contents of all of which are incorporated by reference as if fully set forth herein. This application also relates to U.S. utility application Ser. No. 13/373,081 filed on Nov. 3, 2011 titled “Hydration and Blood Flow Adjusted Glucose Measurement” the contents of which is incorporated by reference as if fully set forth herein. This application also relates to U.S. provisional application 61/926,350 filed on Jan. 12, 2014 titled “Differential Wavelength OCT Analysis System”, U.S. utility application Ser. No. 13/459,168 filed on Apr. 18, 2012 titled “Optic Characteristic Measuring System and Method”, the contents of which are incorporated by reference as if fully set forth herein.

### FIELD OF THE INVENTION

The invention relates to non-invasive optical imaging and analysis of targets including biological tissue structures or components, physiological processes in human body, functionality of the internal organs, using interferometric techniques, such as Optical Coherence Tomography (OCT). In particular, the invention relates to the use of OCT as a very sensitive microphone to non-invasively measure and monitor aspects of a living biological entity. Such aspects of a living biological entity include, but are not limited to: heart activity (heart rate, etc); blood flow; respiration rate; digestive activity; muscle activity; and lactate levels.

### BACKGROUND OF THE INVENTION

Low frequency vibrations in the range of 0.1 Hz to several hundreds of Hz result from physiological processes, such as blood circulation, breathing, and the activity of internal organs. Detection of these vibrations is described in papers such as “Spectral Analysis of Acoustic Vibrations on the Surface of the Human Body” by E. V. Bukhman, et al. (Acoustics Institute, Russian Academy of Sciences, ul. Shvernika 4, Moscow, 117036 Russia) (hereafter referred to as “Spectral Analysis paper”) using detection techniques involving cardiographic transducers and piezoelectric seismic detectors.

Details of typical frequencies associated with different physiological processes are described in the above mentioned Spectral Analysis paper, the contents of which is incorporated herein as if fully set down.

For reliable contact with the body, the detectors used in the above mentioned Spectral Analysis paper needed to be bonded directly to the skin. There is increasing interest in prolonged or continuous monitoring of these physiological processes, or aspects of internal organs for fitness and health monitoring purposes.

The requirement for detectors to be bonded directly to the skin makes this approach unsuitable for prolonged or continuous monitoring of these physiological processes, or aspects of internal organs.

Optical Coherence Tomography (OCT) is a non-invasive imaging and analysis technology that can be used to measure or monitor changes in position of surfaces with great sensitivity. OCT can measure or monitor changes in tissue characteristics in order to measure or monitor specific physiological processes or aspects of internal organs. For example, patent application Ser. No. 13/373,081, filed on Nov. 3, 2011, titled “Hydration and Blood Flow Adjusted Glucose Measurement” and incorporated herein by reference, describes a method of measuring blood flow and other tissue characteristics.

OCT is, however, typically limited to monitoring or measuring characteristics of tissue in regions of tissue that are no more than approximately 3 mm from an accessible surface because of the limited penetration of light into tissue. Accessible surfaces include, but are not limited to the epidermis; the front surface of the cornea of an eye; and, in the case of catheter delivered OCT, the inside surfaces of blood vessels.

There is, therefore, an unmet need for a non-invasive non-contact method and apparatus for detecting the vibrations due to physiological processes, such as those described above, where such physiological processes or aspects of internal organs (heart, lungs, muscles, etc.) can be located more than 3 mm from an accessible surface.

### SUMMARY OF THE INVENTION

The invention described herein meets at least all of the aforementioned unmet needs. The invention provides a method and system for non-invasive imaging and analysis of physiological processes or aspects of internal organs. The invention includes an OCT system that monitors (a) the physical motion of a single tissue structure and (b) the relative motion of at least two tissue structures. Variations with time of the motion or relative motion of such structures are analyzed to determine the frequency content of such variations. Selected components of the frequency spectrum are related to specific physiological processes and aspects of internal organs to yield measurable data. A device according to one embodiment of the invention includes an OCT system, a control module, a processing module, where the OCT system has a fixed inflexible membrane and a flexible membrane so that the positional difference between the two membranes as a consequence of pressure waves corresponding to the human hearing range and also of pressure waves with very low frequency e.g. less than 1 Hz is detected, processed and output. In an alternate embodiment, the measured positional distance is that of a first and a second tissue or membrane component. A variety of alternate embodiments are envisioned, including various OCT system types, and applications include acoustic, as well as bio-sign monitoring and tissue measurement. As used here low frequency means less than 100 Hz.

### BRIEF DESCRIPTION OF THE DRAWINGS

FIG. 1 illustrates of an OCT based microphone according to the invention. FIG. 2 illustrates an example of the OCT based microphone measuring and monitoring the thickness of a blood vessel.

FIG. 3 illustrates an example of the OCT based microphone measuring and monitoring the relative position of other surfaces within a tissue target.

### DETAILED DESCRIPTION OF THE INVENTION

The invention includes an OCT system that monitors the physical motion of one or more structures of a target and derives spectral vibration information from the motion of a single structure or the variation in the relative locations of at least two structures over time. Variations with time of the motion of a single structure or the relative locations of such structures are analyzed to determine the frequency content of such variations. In the particular case of the target being tissue, selected components of the frequency spectrum are related to specific physiological processes and aspects of internal organs to yield measurable data.

In one embodiment the system acts as a conventional microphone in that the target includes a membrane that can physically move in response to a variation in pressure. Conventional microphones, such as those based on a moving coil, ribbon, condenser, electret, or piezo crystal, have negligible response below 10 Hz. Advantages of an OCT based microphone include sensitivity to very low frequency (sub 1 Hz) pressure variations in media such as air, water, or other fluids, and very high dynamic range.

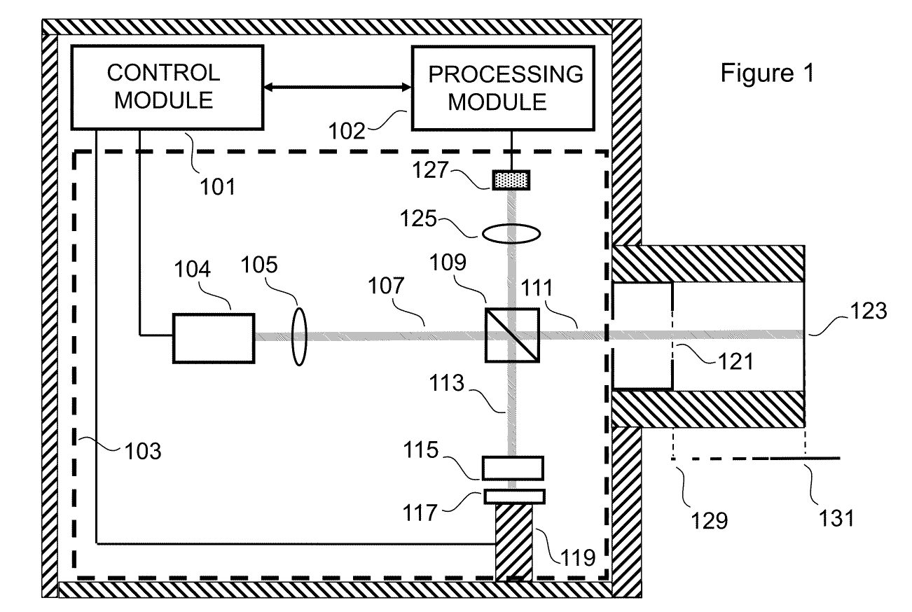

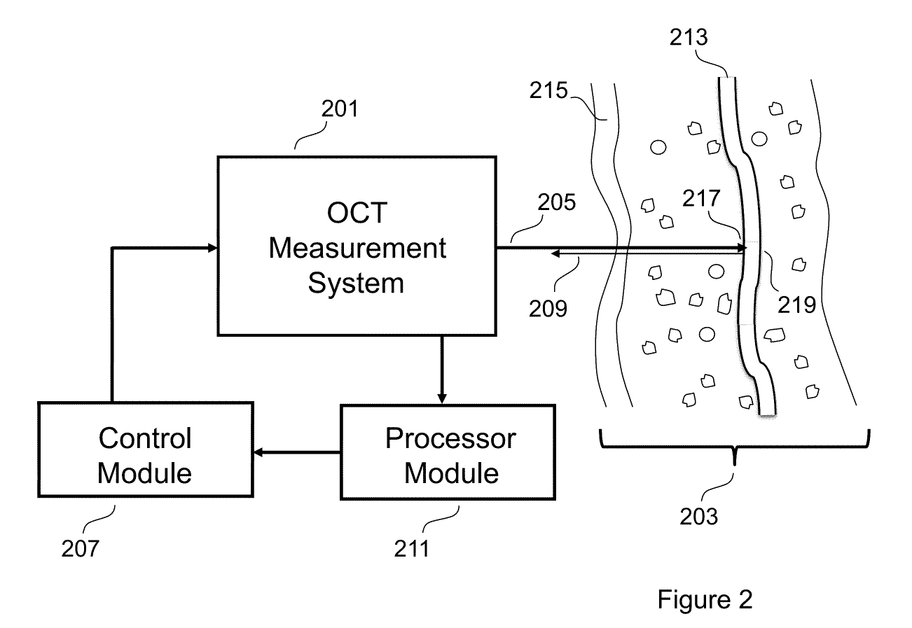

This embodiment is described with respect to FIG. 1 where the OCT system consists of a control module 101, a processing module 102 and an OCT measurement system enclosed in the dashed rectangle 103. The OCT measurement system (of 103) consists of a broadband optical source 104, such as a superluminescent diode. The broadband radiation 107 emitted by the source 104 is collimated by a lens 105 and separated by a beam-splitter 109 into probe radiation 111 and reference radiation 113.

The reference radiation 113 interacts with a partial mirror 115 and a full mirror 117 mounted on a length varying device 119, such as a voice coil or piezo device, to form composite reference radiation that returns to the beam-splitter 109 (as described in U.S. Pat. Nos. 7,526,329 and 7,751,862, incorporated herein by reference).

The probe radiation is directed at the target which in this case consists of a rigid fixed partially reflective surface 121 and a highly reflective flexible membrane surface 123. Both surfaces 121 and 123 reflect some of the probe radiation to the beam-splitter 109. At least a portion of the reflected probe radiation propagates along with the composite reference radiation and is focused by a lens 125 onto a photodiode or detector 127. In an alternate embodiment, the probe radiation is directed at the target that consists of a reflective flexible membrane surface 123 only.

If the flexible membrane surface 123 is exposed to a fluid, such as air or water, the surface 123 can move in response to pressure waves propagating in the fluid. Because the OCT system is measuring the actual physical position of the surface 123 or the relative position of the surface 123 and the fixed rigid (inflexible) surface 121, pressure waves with extremely low frequencies (periods of up to minutes) can be detected and measured.

The detected composite interference signal formed by the interaction of the composite reference radiation and the reflected probe radiation is processed by the processing module 102 to output time varying measurements of the relative positions of the two surfaces 121 and 123 or the position of a single surface 123. This enables the detected interference signals acquired by the OCT system to be processed by the processor module to output electronic signals with sub 1 Hz frequency content. Typically the rigid fixed surface 121 would be aligned with a low order reference signal while the flexible and position varying membrane type surface 123 would be aligned with a high order reference signal that has overlapping adjacent scan segments as indicated by the representation of scan segments 129 and 131. This aspect is further described in U.S. Pat. Nos. 7,526,329, 7,751,862 and 8,605,290, incorporated herein by reference.

An example of an application of this device would be its use as a conventional acoustic microphone (where the fluid is air and the pressure wave is a sound wave), but with a very broad frequency response, including a very low frequency response, and with a very large dynamic range. Another example of an application would be its use as a SONAR detection device (where the fluid is water) and a low frequency response is valuable.

The above embodiment includes a multiple reference OCT system which can simultaneously scan multiple surfaces; however, the embodiment could include SSFD (Swept Source Fourier Domain) or SD (Spectral Domain) or conventional TD (Time Domain) OCT systems, particularly when analyzing low frequency pressure waves. The very low frequency response spectral analysis of the time varying measurement of the relative position of the two surfaces 121 and 123 can be processed for echolocation purposes, seismic exploration, etc.

Motion of the target with respect to the OCT system can be compensated for by a multiple reference OCT system by monitoring the location of at least two surfaces simultaneously. In the case of other OCT systems, motion of the target with respect to the OCT system can be compensated for by scanning at a speed that is significantly faster than the relative motion of the target and the OCT system.

Another embodiment of this invention, where the target is human tissue, is described with respect to FIG. 2, where an OCT measurement system 201 is used to acquire depth scans of a target 203 consisting of tissue. The OCT measurement system 201 is depth aligned, by means of a control module 207, with respect to the tissue target 203 so that probe radiation 205 of the OCT measurement system 201 scans a single surface or structure within the target 203 or at least two surfaces within the target 203.

The back scattered probe radiation 209 that is scattered back to the OCT measurement system 201 where interference signals are acquired and then processed by a processor module 211 to output the position of a single surface or the relative position of at least two surfaces within the target 203. The processor module 211 measures and monitors the position of a single surface or the relative position of at least two surfaces within the target 203 over time.

The variation of the relative position of at least two surfaces within the target 203 over time is further processed by the processor module 211 to provide a spectrum analysis of the variation of the relative position of two surfaces within the target 203. In particular, the extreme low frequency response of an OCT measurement system is exploited to acquire a sub 1 Hz spectrum of the position of a single surface or the relative position of at least two surfaces within the target 203.

Physiological processes and internal organs can generate low frequency pressure waves that modulate the position of a surface or the relative positions of at least two surfaces by either direct interaction between the surface and physiological process or internal organ or by indirect interaction through propagation of pressure waves throughout the body (of which the target tissue is a component).

Low frequency pressure waves are particularly capable of propagating throughout a structure such as a body and are, therefore, capable of modulating a single surface or the relative position of two or more surfaces within the target that can be located at a distance from the source generating the pressure wave.

Referring again to FIG. 2, a blood vessel 213, such as a capillary blood vessel, located close to skin epidermis 215 contains surfaces 217 and 219 suitable for monitoring the flow of blood by the direct interaction of blood flow through the vessel 213 that modulates the relative positions of the surfaces 217 and 219. The process of measuring blood flow in this manner is further described in U.S. utility application Ser. No. 13/373,081.

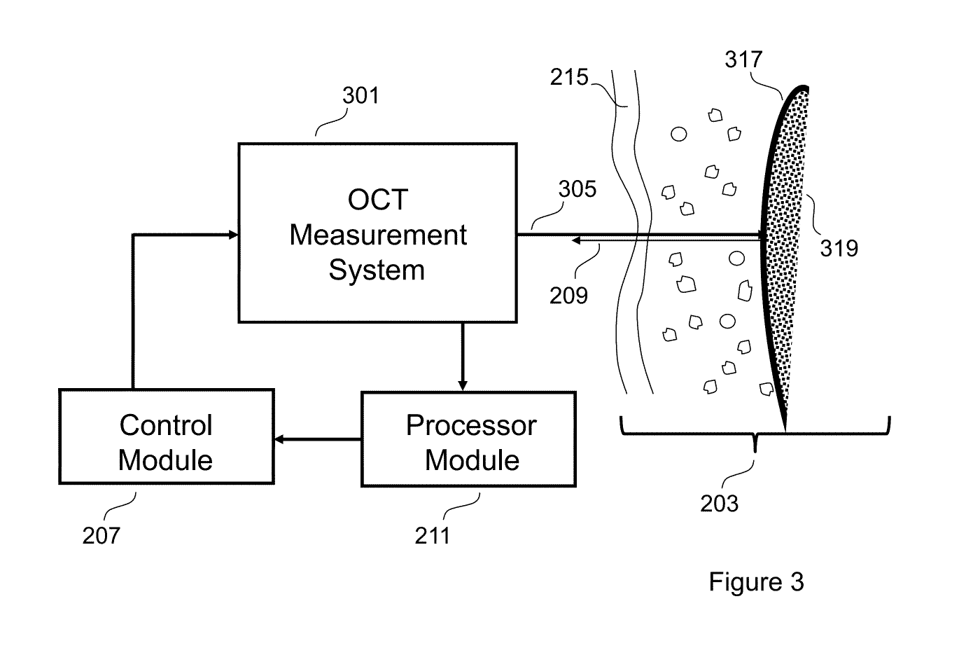

FIG. 3 is in many respects similar to FIG. 2, however, the probe radiation 305 is depth aligned with a different component. In this case the surfaces whose relative positions are modulated are a surface of bone 319 and the periostium layer that typically covers bone. These surfaces are suitable for monitoring pressure waves that propagate throughout the body and may have a source distant from the region of the target being scanned by the OCT measurement system 301.

For example, low frequency pressure waves associated with the respiratory system that propagate throughout the body could be detected by means of an OCT system measuring and monitoring the relative location of surfaces of bone and layers of material physically attached to bone that have different compressibility factors than that of bone.

The processor module 211 of FIGS. 2 and 3 processes the position of a surface or the relative position of at least two surfaces and performs a spectral analysis of the variation in such positional information with time. The module 211 further processes the spectral information to separate information associated with different physiological processes and different internal organs. It then outputs data representing key bio-sign parameters of such physiological processes and internal organs.

Such key bio-sign parameters include, but are not limited to, heart rate; blood pressure; respiratory rate; sounds from muscles or organs or systems such as the digestive system. Values associated with such parameters can be displayed or otherwise made available for routine health monitoring or for optimization of fitness or physical performance.

The above embodiments describe scanning surfaces beneath the epidermis, such as surfaces of blood vessels or of bone, by the OCT measuring system. In addition or instead, other surfaces are used, including, but not limited to, the skin surface, layers of the dermis, and surfaces of the eye, such as surfaces of the cornea or of the crystalline lens.

The OCT measuring system can be a multiple reference OCT system, which simultaneously scans multiple surfaces, or other OCT systems, such as SSFD (Swept Source Fourier Domain) or SD (spectral Domain) or conventional TD (Time Domain).

It can be appreciated that the system and method taught herein may be performed by free space based OCT systems or fiber based OCT systems or those based on wave guides or on micro-bench OCT systems.

The OCT measuring system can be also phase sensitive OCT (H. M. Subhash, N. H. Anh, R. K. K. Wang, S. L. Jacques, N. Choudhury, and A. L. Nuttall, “Feasibility of spectral-domain phase-sensitive optical coherence tomography for middle ear vibrometry,” Journal of Biomedical Optics, vol. 17, Jun 2012.) or nano-sensitive OCT, nsOCT (S. Alexandrov, H. M. Subhash, A. Zam and M. Leahy, “Nano-sensitive optical coherence tomography”, Nanoscale 2014 v.6, pp. 3545-3549).

Other examples will be apparent to persons skilled in the art. The scope of this invention should be determined with reference to the specification, the drawings, the appended claims, along with the full scope of equivalents as applied thereto.

What is claimed is:

1. A microphone device with a sub 100 Hz frequency response, said device comprised of:
a multiple reference OCT system;
a control module; and
a processor module;
wherein, by means of said control module, one reference signal of said OCT system is depth aligned with a fixed rigid surface within a target and a higher order reference signal is depth aligned with a flexible membrane whose position can be modified by a pressure wave propagating within a fluid in contact with said flexible membrane; and
wherein detected interference signals acquired by said OCT system are processed by said processor module to output electronic signals with sub 100 Hz frequency content.

2. A microphone device with a sub 100 Hz frequency response, said device comprised of:
an OCT system;
a control module; and
a processor module;
wherein, by means of said control module, said OCT system scans the relative position of a rigid surface within a target and a flexible membrane whose position can be modified by a pressure wave propagating within a fluid in contact with said flexible membrane; and
wherein detected interference signals acquired by said OCT system are processed by said processor module to output electronic signals with sub 100 Hz frequency content.

3. A microphone device with a sub 100 Hz frequency response, said device comprised of:
an OCT system;
a control module; and
a processor module;
wherein, by means of said control module, said OCT system scans the position of a flexible membrane whose position can be modified by a pressure wave propagating within a fluid in contact with said flexible membrane; and
wherein detected interference signals acquired by said OCT system are processed by said processor module to output electronic signals with sub 100 Hz frequency content.

4. A device for measuring and monitoring bio-sign parameters, said device comprised of:
an OCT system;
a control module; and
a processor module;
wherein, by means of said control module, said OCT system scans the relative position of two or more surfaces within a tissue target;
and wherein the relative position of said two or more surfaces within said tissue target is measured and monitored over time by a processor module;
and wherein the relative position of said two or more surfaces within said tissue target that is measured and monitored over time is further processed to generate a spectrum;
and wherein said spectrum is further processed to separate out signals associated with different bio-sign parameters;
and wherein values associated with different bio-signs parameters are output.

5. A device for measuring and monitoring bio-sign parameters, said device comprised of:
an OCT system;
a control module; and
a processor module;
wherein, by means of said control module, said OCT system scans the relative position of at least one scatterer within a tissue target;
and wherein the relative position of said scatterer within said tissue target and reference mirror of the OCT system is measured and monitored over time by a processor module;
and wherein the relative position of said scatterer within said tissue target that is measured and monitored over time is further processed to generate a spectrum;
and wherein said spectrum is further processed to separate out signals associated with different bio-sign parameters;
and wherein values associated with different bio-signs parameters are output.
